(19) United States
(12) Patent Application Publication
(10) Pub. No.: US 2010/0137576 A1
(43) Pub. Date: Jun. 3, 2010

(54) 5' O [(N ACYL)AMIDOPHOSPHATE] AND 5' O [(N ACYL)AMIDOTHIOPHOSPHATE] AND 5' O [(N ACYL)AMIDODITHIOPHOSPHATE] AND 5' O [(N ACYL)AMIDOSELENOPHOSPHATE] DERIVATIVES OF NUCLEOSIDES AND PROCESSES FOR THE MANUFACTURE THEREOF

(75) Inventors: Wojciech J. Stec, Ksawerow (PL); Janina Baraniak, Lodz (PL); Renata Kaczmarek, Lodz (PL); Ewa Wasilewska, Swedow (PL); Dariusz Korczynski, Lodz (PL); Katarzyna Pieta, Lodz (PL)

Correspondence Address: BROOKS KUSHMAN P.C., 1000 TOWN CENTER, TWENTY-SECOND FLOOR, SOUTHFIELD, MI, 48075, US

(73) Assignee: CENTRUM BADAN MILEKULARNYCHI MAKROMOLEKULARNYCH, P, LODZ (PL)

(21) Appl. No.: 12/444,774

(22) PCT Filed: Oct. 16, 2007
(86) PCT No.: PCT/PL07/00069

§ 371 Date: Jan. 13, 2010

(30) Foreign Application Priority Data
Oct. 17, 2006 (PL) P-380846

(51) Int. Cl. C07H 19/10

(52) U.S. Cl. 536/26.8

(57) ABSTRACT

The subject of the invention includes 5′-O-[(N-acyl)amidophosphate]- and 5′-O-[(N-acyl)amidothiophosphate]- and 5′-O-[(N-acyl)amidodithiophosphate]- and 5′-O-[(N-acyl)amidoselenophosphate]- derivatives of nucleosides.

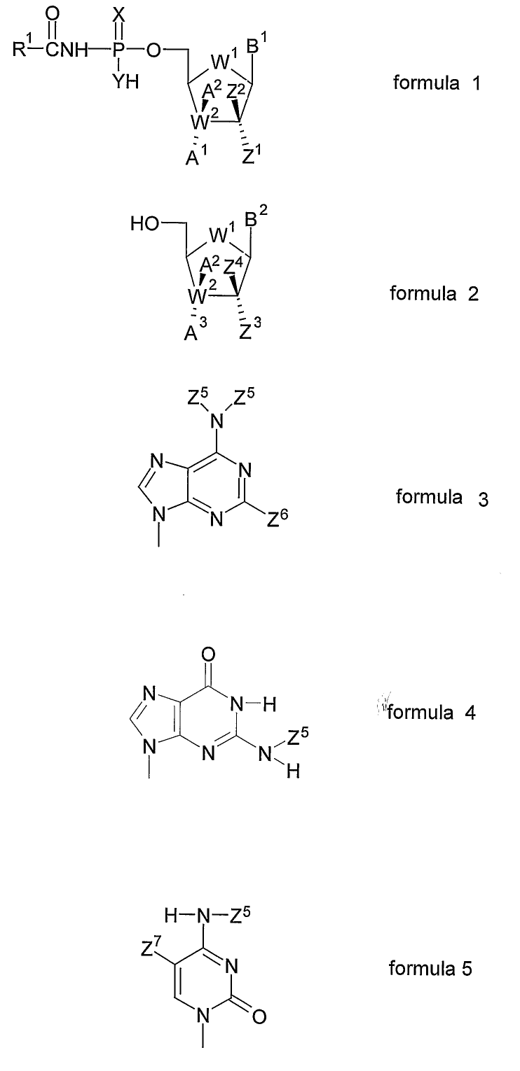

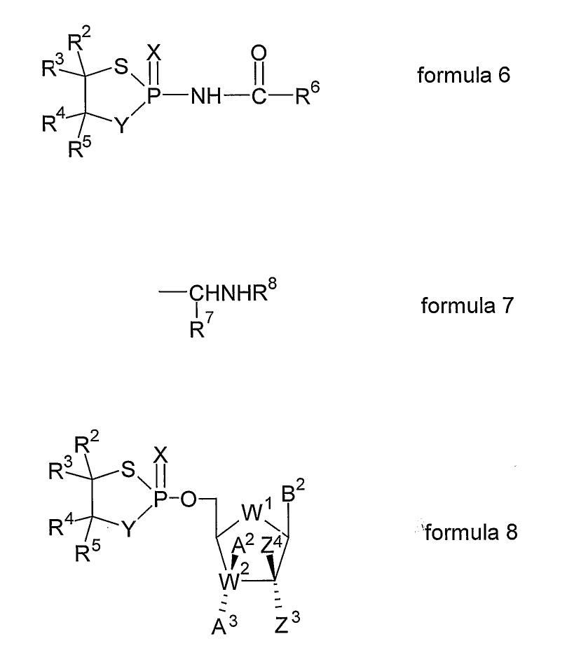

The subject of the invention includes 5′-O-[(N-acyl)amidophosphate]- and 5′-O-[(N-acyl)amidothiophosphate]- and 5′-O-[(N-acyl)amidodithiophosphate]- and 5′-O-[(N-acyl)amidoseleno phosphate]-derivatives of nucleosides of general formula 1 wherein A¹ represents a fluorine atom or azide or hydroxyl group, A² represents a hydrogen atom, B¹ represents an adenine, 2-chloroadenine, 2-bromoadenine, 2-fluoroadenine, 2-iodoadenine, hypoxanthine, guanine, cytosine, 5-fluorocytosine, 5-bromocytosine, 5-iodocytosine, 5-chlorocytosine, azacytosine, thymine, 5-fluorouracil, 5-bromouracil, 5-iodouracil, 5-chlorouracil, 5-(2-bromovinyl)uracil or 2-pyrimidione moiety, W¹ represents an oxygen or carbon atom or a methylidene group, W² represents a carbon atom or W² along with A¹ and A² jointly represent a sulphur or oxygen atom, Z¹ represents a hydrogen or fluorine atom or hydroxyl group, Z² represents a hydrogen or fluorine atom or hydroxyl or methyl group, or Z¹ along with Z² jointly represent a fluoromethylene group, or A¹, A², Z¹ and Z² jointly represent a double bond, X represents an oxygen, sulphur or selenium atom, Y represents an oxygen or sulphur atom, R¹ represents an alkyl or aryl group or a moiety of a primary amino acid amide and the process for the manufacture of 5′-O-[(N-acyl)amidophosphate]- and 5′-O-[(N-acyl)amidothiophosphate]- and 5′-O-[(N-acyl)amidodithiophosphate]- and 5′-O-[(N-acyl)amidoselenophosphate]-derivatives of nucleosides of general formula 1 wherein A¹, A², B¹, W¹, W², Z¹, Z², R¹, X and Y have the meaning mentioned above.

The analogues of purine and pyrimidine nucleosides, such as for example 3′-azido-2′,3′-dideoxythymidine (AZT); 5-fluoro-2′-deoxyuridine (5FdU); 2′,3′-dideoxyinosine (ddI); 2′,3′-dideoxyadenosine (ddA); 2′,3′-dideoxy-2′,3′-didehydrothymidine (d4T); cytarabine (araC, 1-β-D-arabinofuranosylcytosine), gemcitabine (2′-deoxy-2′,2′-difluorocytidine), cladribine (2-chloro-2′-deoxyadenosine), clofarabine (Cl-F-ara-A, 2-chloro-2′-fluoro-2′-deoxy-9-β-D-arabinofuranosyladenine), BVdU [5-(2-bromovinyl)-2′-deoxyuridine], 3TC (2′,3′-dideoxy-3′-thiacytidine), FTC (2′,3′-dideoxy-5-fluoro-3′-thiacytidine), zebularine (1-β-D-ribofuranosyl-2-pyrimidone) constitute an important group of antiviral and anticancer agents.

Nucleoside analogues are taken up by cells owing to the activity of transport proteins specific for their molecules. Having passed the cell membrane barrier, they undergo a three-stage enzymatic phosphorylation which yields 5′-triphosphate derivatives (5′-NTP). A modification of the sugar ring consisting in the replacement of the 2′ or 3′ carbon atom with a heteroatom has a minor influence on the phosphorylation of the nucleosides.

However, the biological activities in a series of nucleosides having the same sugar fragment modification vastly differ depending on nucleobases due to different efficiencies of the metabolic conversion into relevant 5′-triphosphates. The first of the three consecutive phosphorylation processes is crucial, for the nucleoside kinases which catalyse the process are highly substrate-specific depending on the aglycone.

The cytotoxic activity of 5′-NTPs may result from several mechanisms which disrupt either normal DNA and RNA functions or the processes of enzymatic nucleic acid synthesis (Obata, T., Y. Endo, et al. “The molecular targets of antitumor 2′-deoxycytidine analogues.” Curr. Drug. Targets. 2003, 4, 305-13). There are a number of limitations of the direct application of non-modified purine and pyrimidine nucleosides as anticancer and antiviral drugs, such as emergence of resistance to anticancer and antiviral activity resulting from reduced activity of transport proteins (Spratlin, J., R. Sangha, et al. “The absence of human equilibrative nucleoside transporter 1 is associated with reduced survival in patients with gemcitabine-treated pancreas adenocarcinoma.” Clin Cancer Res 2004, 10, 6956-61.) or insufficient phosphorylation activity of thymidine kinase or deoxycytidine kinase (Galmarini, C. M., L. Jordheim, et al. “Pyrimidine nucleoside analogs in cancer treatment”, Expert Rev. Anticancer Ther. 2003, 3, 717-28). To avoid and bypass the difficulties, the current research strategies aiming at a search for more active and effective anticancer and antiviral drugs have been focusing on the preparation of so-called nucleoside prodrugs which use alternative mechanisms of transmembrane transport and intracellular metabolism.

The concept of nucleoside prodrugs consists in the elimination of the first stage of enzymatic phosphorylation by intracellular administration of the substances in the form of monophosphates bound with carriers, typically lipophilic, which facilitate transmembrane transport. After administration, the nucleotides called pronucleotides are expected to undergo chemical and enzymatic transformation in the body so as to produce a target nucleoside monophosphate having a desired pharmacological effect.

The pronucleotide derivatives of anticancer substances and structurally similar antiviral compounds have been the focus of particularly intense research over the last decade. Detailed information about the rapidly developing field of contemporary medicinal chemistry can be found in a number of exhaustive reviews (Parang, K., L. I. Wiebe, et al. “Novel approaches for designing 5′-O-ester prodrugs of 3′-azido-2′, 3′-dideoxythymidine (AZT).” Curr Med Chem 2000, 7, 995-1039.; Peyrottes, S., D. Egron, et al. “SATE pronucleotide approaches: an overview.” Mini Rev. Med. Chem. 2004, 4, 395-408).

The majority of physiological degradation strategies related to the pronucleotides studied so far have been based on the assumption that non-specific enzymes, such as phosphodiesterases and carboxyesterases, induce the release of a drug substance from the pronucleotide by the elimination of one or two protecting groups from the 5′-phosphate moiety. Carboxyesterases have been attractive as carboxymethyl group hydrolases. This activation mechanism was the rationale behind the design of prodrugs with nucleoside amidophosphate structures (carboxymethoxyamino acid derivatives). (Cahard, D.; McGuigan, C.; Balzarini, J. “Aryloxy Phosphoramidate Triesters as Pro-Tides” Mini-Rev. Med. Chem 2004, 4, 371-382; Wagner, C. R.; Iyer, V.v.; McIntee, E. J. “Pronucleotides: towards the in vivo Delivery of Antiviral and Anticancer Nucleotides” Med. Res. Rev. 2000, 20, 417-451).

Those compounds have been selected on the assumption that enzymatic release of the carboxyl group will initiate the intramolecular catalytic cleavage of the phosphorus-nitrogen bond.

In the context of the present patent application it is noted that there are a number of mechanisms for the protection and deprotection of phosphate groups. According to our literature (Chemical Abstract and PubMed) and patent (Delphion) search, the synthesis of 5′-O-[(N-acyl)amido(thio)(dithio)(seleno)phosphate]-derivatives of nucleosides as prodrug nucleoside derivatives with anticancer and antiviral activity has not been reported. Owing to the presence of the P—N bond, the compounds are more prone to the action of phosphoramidases, and the release in the cell of a respective nucleoside-5′-O-phosphate makes it possible to bypass the most restrictive stage of the first enzymatic phosphorylation. Subsequent phosphorylation stages effected by respective kinases lead to the conversion to the target nucleoside-5′-O-triphosphate.

The first synthesis of N-acylamidophosphates was reported in 1962 as a result of the direct esterification of N-acylamidophosphate acids (Zioudrou C. “Reaction of N-acylphosphoamidic acid with alcohols” Tetrahedron, 1962, 18, 197-204). Several years later N-acylamidophosphates were prepared in the reaction of trialkyl phosphite with N-halogenoamides (Desmarchelier J., M.; Fukuto T. R. “Reaction of trialkyl phosphites with haloamides.” J. Org. Chem. 1972, 37, 4218-4220). An alternative synthetic pathway for this class of compounds was a reaction of the carboxamide anion with chlorophosphate (Mizrahi V., Modro T. A. “Phosphoric carboxylic imides. I. Preparation and fragmentation behaviour of dialkylphosphoryl (and phosphinyl)acetyl (and benzoyl) imides and related systems.” J. Org. Chem. 1982, 47, 3533-3539).

None of those reactions, however, was universal, and their common feature was a low yield of desired products. The direct acylation of amidophosphates seemed to be the simplest synthetic method for N-acylated amidophosphates. Unfortunately, the reaction proceeded with the cleavage of the P—N bond in N-acylated amidophosphates and formation of carboxamides.

In 1995 (Robles J.; Pedroso E.; Grandas A. “Peptide-Oligonucleotide Hybrids with N-Acylphosphoramidate Linkages” J. Org. Chem. 1995, 60, 4856-4861), based on amidophosphite chemistry, peptide conjugates with oligonucleotides containing an N-acylamidophosphate bond were synthesised. Aminoacyladenylates were prepared following a similar approach in 2000 (Moriguchi T.; Yanagi T.; Kunimori M.; Wada T.; Sekine M. “Synthesis and Properties of Aminoacylamido-AMP: Chemical Optimization for the Construction of an N-Acyl Phosphoramidate Linkage” J. Org. Chem. 2000, 65, 8229-8238).

5′-O-[(N-acyl) amidophosphate]- and 5-O-[(N-acyl)amidothiophosphate]- and 5′-O-[(N-acyl)amidodithiophosphate]- and 5′-O-[(N-acyl)amidoselenophosphate]-derivatives of nucleosides of general formula 1 wherein A¹ represents a fluorine atom or azide or hydroxyl group, A² represents a hydrogen atom, B¹ represents an adenine, 2-chloroadenine, 2-fluoroadenine, 2-bromoadenine, 2-iodoadenine, hypoxanthine, guanine, cytosine, 5-fluorocytosine, 5-bromocytosine, 5-iodocytosine, 5-chlorocytosine, azacytosine, thymine, 5-fluorouracil, 5-bromouracil, 5-iodouracil, 5-chlorouracil, 5-(2-bromovinyl)uracil or 2-pyrimidione moiety, W¹ represents an oxygen or carbon atom or a methylidene group, W² represents a carbon atom or W² along with A¹ and A² jointly represent a sulphur or oxygen atom, Z¹ represents a hydrogen or fluorine atom or hydroxyl group, Z² represents a hydrogen or fluorine atom or hydroxyl or methyl group, or Z¹ along with Z² jointly represent a fluoromethylene group, or A¹, A², Z¹ and Z² jointly represent a double bond, X represents an oxygen, sulphur or selenium atom, Y represents an oxygen or sulphur atom, R¹ represents a simple alkyl or aryl group with 1-6 carbon atoms or a moiety of a primary amino acid amide.

The process for the manufacture of 5′-O-[(N-acyl)amidophosphate]- and 5′-O-[(N-acyl)amidothiophosphate]- and 5′-O-[(N-acyl)amidodithiophosphate]- and 5′-O-[(N-acyl)amidoselenophosphate]-derivatives of nucleosides of general formula 1 wherein A¹, A², B¹, R¹, W², Z¹, Z², X and Y are as above according to the present invention consists in that a nucleoside of general formula 2 wherein A², W¹ are as above, A³ represents a fluorine atom or azide or protected hydroxyl group, W² represents a carbon atom or A², A³ and W² jointly represent a sulphur or oxygen atom, B² represents an adenine, 2-chloroadenine, 2-fluoroadenine, 2-bromoadenine, 2-iodoadenine, hypoxanthine, guanine or cytosine moiety of formulas 3, 4, 5 wherein Z⁵ represents a hydrogen atom or a known exoamine blocking group, Z⁶ represents a hydrogen atom or a chlorine, fluorine, bromine or iodine atom, Z⁷ represents a hydrogen atom or fluorine, chlorine, bromine or iodine atom or B² represents a thymine moiety, an azacytosine moiety or a 5-fluorouracil, 5-bromouracil, 5-iodouracil, 5-chlorouracil, 5-(2-bromovinyl)uracil or 2-pyrimidione moiety, and Z³ represents a hydrogen or fluorine atom or a protected hydroxyl group, Z⁴ represents a hydrogen or fluorine atom, a protected hydroxyl group or a methyl group or Z³ and Z⁴ jointly represent a fluoromethylene group or A², A³, Z³, Z⁴ jointly represent a double bond, is condensed with a compound of general formula 6, wherein R², R³, R⁴ and R⁵ each represent a hydrogen atom, simple alkyl or aryl with 1-6 carbon atoms, R⁶ represents an aromatic or non-aromatic 5- or 6-membered heterocyclic ring with 1-3 heteroatoms selected from a group consisting of an oxygen atom, nitrogen atom and sulphur atom, wherein the aryl or heterocyclic groups may be substituted with 1, 2 or 3 substituents independently selected from a group consisting of alkyl, halogen atom CHF₂, CF₃, alkoxyl, halogenoalkoxyl, alkylthio group or R⁶ represents alkyl or phenyl or cycloalkyl which may be substituted with 1, 2 or 3 substituents independently selected from a group consisting of alkyl, alkenyl, alkynyl, alkoxyl, alkenyloxyl, alkynoxyl, cycloalkyl, cycloalkenyl, cycloalkyloxyl, cycloalkenyloxyl, phenyl and chlorine atom, and the phenyl may be substituted with 1-5 halogen atoms and/or 1-3 substituents independently selected from a group consisting of alkyl, halogenoalkyl, alkoxyl, halogenoalkoxyl, alkylthio group or halogenoalkylthio group, wherein the phenylamide group condensed with a saturated or unsaturated 5- or 6-membered ring which may be substituted with one or more alkyl groups and/or possibly containing a heteroatom selected from a group consisting of an oxygen atom, a nitrogen atom and a sulphur atom, or R⁶ represents a moiety of a primary amino acid amide of general formula 7 wherein R⁷ represents an amino acid side chain, R⁸ represents a hydrogen atom or a known amino acid alpha-amine-blocking group, X represents an oxygen, sulphur or selenium atom, Y represents an oxygen or sulphur atom; the condensation is carried out in anhydrous organic solvents in the presence of condensation activators, and after reaction completion the amino acid alpha-amine-blocking group, the 2′- and 3′-hydroxyl blocking groups and the nucleoside exoamine blocking groups are removed using methods known in the art.

The protecting groups used for the 2′- and 3′-hydroxyl groups preferably include known protecting groups selected from a group consisting of the acyl, benzoyl, 4,4-dimethoxytriphenyl, benzyl, trialkylsilyl, in particular trimethylsilyl group.

The protecting groups used for the exoamine groups preferably include known exoamine protecting groups selected from a group consisting of the phenoxyacetyl, isopropoxyacetyl, isobutyryl, benzoyl, (dialkylamino)methylene and (dialkylamino)ethylidene group.

The protecting groups used for the amino acid alpha-amine groups preferably include known alpha-amine protecting groups selected from a group consisting of the acyl, trifluoroacetyl, 4,4-dimethoxytriphenyl, benzyloxycarbonyl and tert-butyloxycarbonyl group.

The condensation activators used include non-nucleophilic alcoholates, such as potassium tert-butanolate, or amines, such as imidazole, 1-methylimidazole, 4-dimethylaminopyridine, triethylamine and in particular 1,8-diazabicyclo[5.4]undec-7-ene (DBU).

The condensation reaction is preferably carried out in an anhydrous organic solvent selected from a group consisting of acetonitrile, methylene chloride, N,N-dimethylformamide, pyridine, dioxane and tetrahydrofurane.

In the process according to the present invention, compounds of formula 1, wherein X and Y represent an oxygen atom, are preferably obtained from previously prepared compounds of formula 1, wherein X═S, Y═S or Y═O, or X═Se and Y═O in the oxidation reaction using an oxidation reagent known in the art, particularly hydrogen peroxide.

The process for the manufacture of 5′-O-[(N-acyl)amidophosphate]- and 5′-O-[(N-acyl)amidothiophosphate]- and 5′-O-[(N-acyl)amidodithiophosphate]- and 5′-O-[(N-acyl)amidoselenophosphate]-derivatives of nucleosides of general formula 1 wherein A¹, A², B¹, R¹, W², W², Z1¹ Z², X and Y are as above according to the present invention consists in that the reagents subject to the condensation reaction include primary amides of carboxylic acids of general formula R⁶CONH₂, wherein R⁶ is as above or amino acid amides with moieties of formula 7, wherein R⁷ and R⁸ are as above, with nucleoside derivatives of general formula 8, wherein A², A³, B², R², R³, R⁴, R⁵, W¹, W², Z³ and Z⁴ are as above, X represents an oxygen, sulphur or selenium atom, Y represents an oxygen or sulphur atom, and the condensation reaction is carried out in anhydrous organic solvents in the presence of condensation activators, and after reaction completion the amino acid alpha-amine-blocking groups, the 2′- and 3′-hydroxyl blocking groups and the nucleoside exoamine blocking groups are removed using methods known in the art.

The protecting groups for the 2′- and 3′-hydroxyl groups preferably include known protecting groups selected from a group consisting of the acyl, benzoyl, 4,4-dimethoxytriphenyl, benzyl, trialkylsilyl and in particular trimethylsilyl group.

The protecting groups used for the exoamine groups preferably include known protecting groups selected from a group consisting of the phenoxyacetyl, isopropoxyacetyl, isobutyryl, benzoyl, (dialkylamino)methylene and (dialkylamino)ethylidene group.

The protecting groups used for the amino acid alpha-amine groups include known alpha-amine protecting groups preferably selected from a group consisting of the acyl, trifluoroacetyl, 4,4-dimethoxytriphenyl, benzyloxycarbonyl and tert-butyloxycarbonyl group.

The condensation activators used include non-nucleophilic alcoholates, such as potassium tert-butanolate, or amines, such as imidazole, 1-methylimidazole, 4-dimethylaminopyridine, triethylamine and in particular 1,8-diazabicyclo[5.4]undec-7-ene (DBU).

The condensation reaction is preferably carried out in an anhydrous organic solvent selected from a group consisting of acetonitrile, methylene chloride, N,N-dimethylformamide, pyridine, dioxane and tetrahydrofurane.

In the process according to the present invention, compounds of formula 1, wherein X and Y represent an oxygen atom, are preferably obtained from previously prepared compounds of formula 1 wherein X═S, Y═S or Y═O, or X═Se and Y═O in the oxidation reaction using an oxidation reagent known in the art, particularly hydrogen peroxide. The process according to the present invention is general and may be used in the direct synthesis of N-acylamidophosphates of general formula 1.

The process according to the invention is used in the manufacture of 5′-O-[(N-acyl)amidophosphate]- and 5′-O-[(N-acyl)amidothiophosphate]- and 5′-O-[(N-acyl)amidodithiophosphate]- and 5′-O-[(N-acyl)amidoselenophosphate]-derivatives of nucleosides of general formula 1 wherein A¹ represents a fluorine atom or azide or hydroxyl group, A² represents a hydrogen atom, B¹ represents an adenine, 2-chloroadenine, 2-fluoroadenine, 2-bromoadenine, 2-iodoadenine, hypoxanthine, guanine, cytosine, 5-fluorocytosine, 5-bromocytosine, 5-iodocytosine, 5-chlorocytosine, azacytosine, thymine, 5-fluorouracil, 5-chlorouracil, 5-bromouracil, 5-iodouracil, 5-(2-bromovinyl)uracil or 2-pyrimidione moiety, W¹ represents an oxygen or carbon atom or a methylidene group, W² represents a carbon atom or A¹, A² and W² jointly represent a sulphur or oxygen atom, Z¹ represents a hydrogen or fluorine atom or hydroxyl group, Z² represents a hydrogen or fluorine atom or hydroxyl or methyl group, or Z¹ along with Z² jointly represent a fluoromethylene group, or A¹, A², Z¹ and Z² jointly represent a double bond, X represents an oxygen, sulphur or selenium atom, Y represents an oxygen or sulphur atom, R¹ represents a simple alkyl or aryl group with 1-6 carbon atoms or a moiety of a primary amino acid amide.

The process according to the present invention is illustrated in the examples which follow.

### EXAMPLE 1

### Gemcitabine-5′-O-(N-benzoyl)amidothiophosphate

To a solution of 1 mmol of N,O^{3′}-dibenzoylgemcitabine in 10 mL of methylene chloride 1 mmol of DBU was added. Subsequently a solution of 1 mmol of N-(2-thiono-1,3,2-oxathiaphospholanyl)benzamide in 5 mL of CH₂Cl₂ was added dropwise. The reaction was carried out at 40° C. for 48 hours (TLC and ³¹P NMR analyses). The reaction mixture was then concentrated under reduced pressure and aqueous saturated ammonia (30 mL) was added to the residue (ambient temperature, 48 hours). The ammonia was subsequently distilled off under reduced pressure. The product was isolated in a 56% yield using ion-exchange chromatography (DEAE-Sephadex A-25) with TEAB (0.0→0.25M; pH=7.5) as the eluent. ³¹P NMR (CH₃OD) δ: 46.8, 47.5 ppm. FAB-MS m/z: (M+1) 463.

### EXAMPLE 2

### 3′-Azido-5′-O-[N-(carbonyl-4-pyridine)]amidophosphorano-3′-deoxythymidine

To 1 mmol of N-(2-oxo-1,3,2-oxathiaphospholanyl)isonicotinamide dissolved in 5 mL of CH₃CN a mixture of 1 mmol of AZT dissolved in 7 mL of CH₃CN and 1 mmol of DBU was added. The reaction was carried out at ambient temperature for 24 hours. The resulting product was isolated from the reaction mixture in a 48% yield using column chromatography with 230-400 mesh silica gel and a chloroform:methanol:water (10:6:1) mixture as the eluent; ³¹P NMR (D₂O) δ: −4.2 ppm. FAB-MS m/z: (M−1) 451.

### EXAMPLE 3

### Cytarabine-5′-O-(N-acetyl)amidodithiophosphate

To a solution of 1 mmol of acetamide in 8 mL of N,N-dimethylformamide 1 mmol of DBU was added. Subsequently a solution of N,O^{2′},O^{3′}-tribenzoylcytarabine-N-(2-thiono-1,3,2-dithiaphospholate) in 3 mL of DMF was added dropwise. The reaction was carried out at ambient temperature for 20 hours. The reaction mixture was then concentrated under reduced pressure and aqueous saturated ammonia (30 mL) was added to the residue (ambient temperature, 48 hours). The ammonia was subsequently distilled off under reduced pressure. The product was isolated from the reaction mixture in a 35% yield using ion-exchange chromatography (DEAE-Sephadex A-25) with TEAB (0.0→0.5M; pH=7.5) as the eluent. ³¹P NMR (D₂O) δ: 103.2 ppm. FAB-MS m/z: (M−1) 395.2.

### EXAMPLE 4

### Clofarabine-5′-O-(N-Prolyloamido)amidoselenophosphate

To a solution of 1 mmol of N⁶,N⁶,O^{3′}-tribenzoylclofarabine in 10 mL of pyridine 1 mmol of DBU was added. Subsequently a solution of 1 mmol of N-(2-seleno-1,3,2-oxathiaphospholanyl)-N-α-dimethoxytrityl-prolinamide in 5 mL of CH₃CN was added dropwise. The reaction was carried out at ambient temperature for 12 hours (TLC and ³¹P NMR analyses). The reaction mixture was then concentrated under reduced pressure and aqueous saturated ammonia (30 mL) was added to the residue (ambient temperature, 48 hours). The ammonia was then distilled off under reduced pressure and a solution of trifluoroacetic acid in CH₂Cl₂ (1:1, 30 mL) was added to the residue; the solution was agitated for further 30 min. The product was isolated in a 52% yield using ion-exchange chromatography (DEAE-Sephadex A-25) with TEAB (0.0→0.25M; pH=7.5) as the eluent. ³¹P NMR (CH₃OD) δ: 43.1 ppm. FAB-MS m/z: (M−1) 542.2

### EXAMPLE 5

### Gemcitabine-5′-O-(N-phenylacetyl)amidothiophosphate

To a solution of 1 mmol of N,O^{3′}-dibenzoylgemcitabine in 10 mL of methylene chloride 1 mmol of DBU was added. Subsequently a solution of 1 mmol of N-(2-thiono-1,3,2-oxathiaphospholanyl)phenylacetamide in 5 mL of CH₂Cl₂ was added dropwise. The reaction was carried out at 40° C. for 60 hours. The reaction mixture was then concentrated under reduced pressure and aqueous saturated ammonia (30 mL) was added to the residue (ambient temperature, 48 hours). The ammonia was subsequently distilled off under reduced pressure. The product was isolated in a 52% yield using ion-exchange chromatography (DEAE-Sephadex A-25) with TEAB (0.0→0.25M; pH=7.5) as the eluent. ³¹P NMR (CH₃OD) δ: 46.8, 47.5 ppm. FAB-MS m/z: (M−1) 475.

### EXAMPLE 6

### Zebularine-5′-O-[N-(2-hydroxybenzoyl)]amidophosphate

To 1 mmol of N-(2-oxo-1,3,2-oxathiaphospholanyl)-2-acetyloxybenzamide dissolved in 5 mL of CH₃CN a mixture of 1 mmol of 2′,3′-dibenzoylzebularine dissolved in 10 mL of CH₃CN and 1 mmol of DBU was added. The reaction was carried out at ambient temperature for 24 hours. The reaction mixture was then concentrated under reduced pressure and aqueous saturated ammonia (20 mL) was added to the residue (ambient temperature, 2 hours). The ammonia was subsequently distilled off under reduced pressure. The product was isolated from the reaction mixture in a 60% yield using ion-exchange chromatography (DEAE-Sephadex A-25) with TEAB (0.0→0.3M, pH=7.5) as the eluent. ³¹P NMR (D₂O) δ: −4.9 ppm. FAB-MS m/z: (M−1) 426.2.

### EXAMPLE 7

### Azacytidine-5′-O-[N-(S)-2-amino-3-phenyl-propionocarbonyl]amidophosphate

To a solution of 1 mmol of N,2′,3′-tribenzoylazacytidine in 10 mL of acetonitrile 1 mmol of DBU was added. Subsequently a solution of 1 mmol of N-(2-oxo-1,3,2-oxathiaphospholanyl)-(N-α-Boc-phenylalanylamide) in 5 mL of CH₃CN was added dropwise. The reaction was carried out at 30° C. for 36 hours. The reaction mixture was then concentrated under reduced pressure and aqueous saturated ammonia (30 mL) was added to the residue (ambient temperature, 48 hours). The ammonia was then distilled off under reduced pressure and a solution of trifluoroacetic acid in CH₂Cl₂ (1:1, 30 mL) was added to the residue; the solution was agitated for further 30 min. The product was isolated in a 44% yield using ion-exchange chromatography (DEAE-Sephadex A-25) with TEAB (0.0→0.20M; pH=7.5) as the eluent. ³¹P NMR (CH₃OD) δ: −5.0 ppm. FAB-MS m/z (M+1) 471.2.

### EXAMPLE 8

### Troxacitabine-5′-O-[2-(6-methoxy-2-naphthyl)propanecarbonyl]amidophosphate

To 1 mmol of N-(2-oxo-1,3,2-oxathiaphospholanyl)-2-(6-methoxy-2-naphthyl)propanamide dissolved in 5 mL of dioxane a mixture of 1 mmol of troxacitabine dissolved in 7 mL of CH₃CN and 1 mmol of DBU was added. The reaction was carried out at ambient temperature for 24 hours. The resulting product was isolated from the reaction mixture in a 60% yield using column chromatography with 230-400 mesh silica gel and a chloroform:methanol:water (9:6:0.5) mixture as the eluent; ³¹P NMR (D₂O) δ: −4.2 ppm; FAB-MS m/z: (M−1) 503.4.

### EXAMPLE 9

### Troxacitabine-5′-O-[2-(6-methoxy-2-naphthyl)propanecarbonyl]amidodithiophosphate

To 1 mmol of N-(2-thiono-1,3,2-dithiaphospholanyl)-2-(6-methoxy-2-naphthyl)propanamide dissolved in 5 mL of acetonitrile a mixture of 1 mmol of troxacitabine dissolved in 7 mL of CH₃CN and 1 mmol of DBU was added. The reaction was carried out at ambient temperature for 24 hours. The resulting product was isolated from the reaction mixture in a 53% yield using column chromatography with 230-400 mesh silica gel and a chloroform:methanol:water (9:6:0.5) mixture as the eluent; ³¹P NMR (D₂O) δ: 105.4 ppm; FAB-MS m/z: (M−1) 535.2.

### EXAMPLE 10

### Clofarabine-5′-O-(N-trifluoroacetyl)amidodithiophosphate

To a solution of 1 mmol of trifluoroacetamide in 8 mL of methylene chloride 3 mmol of imidazole was added. Subsequently a solution of 1 mmol of N,O^{3′}-dibenzoylclofarabine-N-(2-thiono-1,3,2-dithiaphospholate) in 3 mL of methylene chloride was added dropwise. The reaction was carried out at ambient temperature for 48 hours. The reaction mixture was then concentrated under reduced pressure and aqueous saturated ammonia (30 mL) was added to the residue (ambient temperature, 48 hours). The ammonia was subsequently distilled off under reduced pressure. The product was isolated from the reaction mixture in a 42% yield using ion-exchange chromatography (DEAE-Sephadex A-25) with TEAB (0.0→0.6M; pH=7.5) as the eluent. ³¹P NMR (D₂O) δ: 104.3 ppm. FAB-MS m/z: (M−1) 455.70.

### EXAMPLE 11

### Tezacitabine-5′-O-(N-trifluoroacetyl)amidophosphate

To a solution of 1 mmol of trifluoroacetamide in 10 mL of tetrahydrofurane 3 mmol of imidazole was added. Subsequently a solution of 1 mmol of N,O^{3′}-diisopropoxyacetyltezacytabine-N-(2-thiono-1,3,2-dithiaphospholate) in 5 mL of tetrahydrofurane was added dropwise. The reaction was carried out at ambient temperature for 48 hours. The reaction mixture was then concentrated under reduced pressure and aqueous saturated ammonia (30 mL) was added to the residue (ambient temperature, 2 hours). The ammonia was subsequently distilled off under reduced pressure. The product was isolated from the reaction mixture in a 38% yield using ion-exchange chromatography (DEAE-Sephadex A-25) with TEAB (0.0→0.45M; pH=7.5) as the eluent. ³¹P NMR (D₂O) δ: −3.8 ppm; FAB-MS m/z: (M−1) 377.3.

### EXAMPLE 12

### Clofarabine-5′-O-[N-[2-(1,3-dioxo-1,3-dihydro-2H-isoindol-2-yl)acetyl]amidothiophosphate

To a solution of 1 mmol of N⁶,N⁶,O^{3′}-tribenzoylclofarabine in 10 mL of tetrahydrofurane 1 mmol of DBU was added. Subsequently a solution of 1 mmol of N-(2-thiono-1,3,2-oxathiaphospholanyl)-2-(1,3-dioxo-1,3-dihydro-2H-isoindol-2-yl)acetamide in 5 mL of tetrahydrofurane was added dropwise. The reaction was carried out at ambient temperature for 12 hours (TLC and ³¹P NMR analyses). The reaction mixture was then concentrated under reduced pressure and aqueous saturated ammonia (30 mL) was added to the residue (ambient temperature, 48 hours). The ammonia was subsequently distilled off under reduced pressure. The product was isolated in a 40% yield using ion-exchange chromatography (DEAE-Sephadex A-25) with TEAB (0.0→0.35M; pH=7.5) as the eluent. ³¹P NMR (CH₃OD) δ: 47.1 ppm; 47.3 ppm. FAB-MS m/z: (M−1) 585.7.

### EXAMPLE 13

### Cladribine-5′-O-[N-[2-(1,3-dioxo-1,3-dihydro-2H-isoindol-2-yl)acetyl]amidoselenophosphate

To a solution of 1 mmol of N⁶,N⁶,O^{3′}-triacetylcladribine in 10 mL of acetonitrile 1 mmol of DBU was added. Subsequently a solution of 1 mmol of N-(2-seleno-1,3,2-oxathiaphospholanyl)-2-(1,3-dioxo-1,3-dihydro-2H-isoindol-2-yl)acetamide in 5 mL of acetonitrile was added dropwise. The reaction was carried out at ambient temperature for 12 hours (TLC and ³¹P NMR analyses). The reaction mixture was then concentrated under reduced pressure and aqueous saturated ammonia (30 ml mL) was added to the residue (ambient temperature, 48 hours). The ammonia was subsequently distilled off under reduced pressure. The product was isolated in a 35% yield using ion-exchange chromatography (DEAE-Sephadex A-25) with TEAB (0.0→0.4M; pH=7.5) as the eluent. ³¹P NMR (CH₃OD) δ: 42.8 ppm, 42.95 ppm; FAB-MS m/z: (M−1) 613.9

### EXAMPLE 14

### Troxacitabine-5′-O-[N-(S)-2-aminopropionocarbonyl]amidophosphate

To a solution of 1 mmol of troxacitabine in 10 mL of pyridine 4 mmol of 4-dimethylaminopyridine was added. Subsequently a solution of 1 mmol of N-(2-oxo-1,3,2-oxathiaphospholanyl)-(N-α-dimethoxytriphenyl-alanylamide) in 5 mL of CH₃CN was added dropwise. The reaction was carried out at ambient temperature for 40 hours. The reaction mixture was concentrated under reduced pressure and a solution of trifluoroacetic acid in CH₂Cl₂ (1:1, 30 mL) was added to the residue; the solution was agitated for further 30 min. The product was isolated in a 30% yield using ion-exchange chromatography (DEAE-Sephadex A-25) with TEAB (0.0→0.20M; pH=7.5) as the eluent. ³¹P NMR (CH₃OD) δ: −5.4 ppm. FAB-MS m/z: (M−1) 362.3.

### EXAMPLE 15

### Cladribine-5′-O-[N-(S)-2-aminopropionocarbonyl]amidothiophosphate

To a solution of 1 mmol of O^{3′}-acylcladribine in 10 mL of acetonitrile 4 mmol of 1-methylimidazole was added. Subsequently a solution of 1 mmol of N-(2-thiono-1,3,2-oxathiaphospholanyl)-(N-α-Boc-alanylamide) in 5 mL of CH₃CN was added dropwise. The reaction was carried out at ambient temperature for 40 hours. The reaction mixture was concentrated under reduced pressure and aqueous saturated ammonia (20 mL) was added to the residue (ambient temperature, 2 hours). Thereafter the reaction mixture was again concentrated and a solution of trifluoroacetic acid in CH₂Cl₂ (1:1, 30 mL) was added; the solution was agitated for further 30 min. The product was isolated in a 30% yield using ion-exchange chromatography (DEAE-Sephadex A-25) with TEAB (0.0→0.5M; pH=7.5) as the eluent. ³¹P NMR (CH₃OD) δ: 46.8, 47.5 ppm. FAB-MS m/z: (M−1) 450.7.

### EXAMPLE 16

### Troxacitabine-5′-O-[N-(5-methylisoxazol-3-carbonyl]amidothio phosphate

To a solution of 1 mmol of troxacitabine in 10 mL of pyridine 1 mmol of DBU was added. Subsequently a solution of 1 mmol of N-(2-thiono-1,3,2-oxathiaphospholanyl)-(N-(5-methylisoxazol-3-amide) in 5 mL of pyridine was added dropwise. The reaction was carried out at ambient temperature for 30 hours. The reaction mixture was subsequently concentrated under reduced pressure. The product was isolated in a 50% yield using ion-exchange chromatography (DEAE-Sephadex A-25) with TEAB (0.0->0.4M; pH=7.5) as the eluent. ³¹P NMR (CH₃OD) δ: −45.8 ppm and 46.2 ppm. FAB-MS m/z: (M−1) 416.3.

### EXAMPLE 17

### Clofarabine-5′-O-[N-(4-carbonylpiperidine]amidophosphate

To a solution of 1 mmol of N⁶,N⁶,O^{3′}-tribenzoylclofarabine in 10 mL of acetonitrile 1 mmol of DBU was added. Subsequently a solution of 1 mmol of N-(2-oxo-1,3,2-oxathiaphospholanyl)piperidine-4-carboxamide in 5 mL of acetonitrile was added dropwise. The reaction was carried out at ambient temperature for 10 hours (TLC and ³¹P NMR analyses). The reaction mixture was then concentrated under reduced pressure and aqueous saturated ammonia (30 mL) was added to the residue (ambient temperature, 48 hours). The ammonia was subsequently distilled off under reduced pressure. The product was isolated in a 38% yield using ion-exchange chromatography (DEAE-Sephadex A-25) with TEAB (0.0→0.5M; pH=7.5) as the eluent. ³¹P NMR (CH₃OD) δ: −4.8 ppm; FAB-MS m/z: (M−1) 492.7

### EXAMPLE 18

### Gemcitabine-5′-O-[(N-(2-benzo[1,3]dioxol-5-yl-acetyl]amidodithio phosphate

To a solution of 1 mmol of N,O^{3′}-diisobutyrylgemcitabine in 10 mL of DMF 1 mmol of DBU was added. Subsequently a solution of 1 mmol of N-(2-thiono-1,3,2-dithiaphospholanyl)-2-benzo[1,3]-5-yl-acetamide in 5 mL of DMF was added dropwise. The reaction was carried out at ambient temperature for 48 hours (TLC and ³¹PNMR analyses). The reaction mixture was then concentrated under reduced pressure and aqueous saturated ammonia (30 mL) was added to the residue (ambient temperature, 48 hours). The ammonia was subsequently distilled off under reduced pressure. The product was isolated in a 60% yield using ion-exchange chromatography (DEAE-Sephadex A-25) with TEAB (0.0→0.5M, pH=7.5) as the eluent. ³¹P NMR (CH₃OD) δ: 106.1 ppm. FAB-MS m/z: (M−1) 536.1.

### EXAMPLE 19

### 2′,3′-Dideoxy-2′,3′-didehydrothymidine-5′-O-[N-(2-carbonylquinoline]amidothiophosphate

To a solution of 1 mmol of 2′,3′-dideoxy-2′,3′-didehydrothymidine (d4T) in 10 mL of acetonitrile 1 mmol of DBU was added. Subsequently a solution of 1 mmol of N-(2-thiono-1,3,2-oxathiaphospholanyl)quinoline-2-carboxamide in 5 mL of CH₂Cl₂ was added dropwise The reaction was carried out at ambient temperature for 24 hours (TLC and ³¹P NMR analyses). The reaction mixture was concentrated under reduced pressure and the product was isolated in a 68% yield using ion-exchange chromatography (DEAE-Sephadex A-25) with TEAB (0.0→0.35M; pH=7.5) as the eluent. ³¹P NMR (CH₃OD) δ: 44.9, 45.2 ppm. FAB-MS m/z: (M−1) 474.2.

### EXAMPLE 20

### 3′-Azido-5′-O-(N-benzoyl)amidophosphorano-3′-deoxythymidine

3′-Azido-5′-O-(N-benzoyl)amidothiophosphorano-3′-deoxythymidine (1 mmol) was dissolved in 25 mL of 3% hydrogen peroxide and the mixture was agitated at ambient temperature for 1 hour. The water was subsequently distilled off under reduced pressure. The resulting product was isolated from the reaction mixture in an 80% yield using column chromatography with 230-400 mesh silica gel and a chloroform:methanol (7:3) mixture as the eluent; ³¹P NMR (D₂O) δ: −4.6 ppm FAB-MS m/z: (M−1) 449.

1. 5′-O-[(N-acyl)amidophosphate]- and 5′-O-[(N-acyl)amidothiophosphate]- and 5′-O-[(N-acyl)amidodithiophosphate]- and 5′-O-[(N-acyl)amidoselenophosphate]- derivatives of nucleosides of general formula 1 wherein A¹ represents a fluorine atom or azide or hydroxyl group, A² represents a hydrogen atom, B¹ represents an adenine, 2-chloroadenine, 2-fluoroadenine, 2-bromoadenine, 2-iodoadenine, hypoxanthine, guanine, cytosine, 5-fluorocytosine, 5-bromocytosine, 5-iodocytosine, 5-chlorocytosine, azacytosine, thymine, 5-fluorouracil, 5-bromouracil, 5-iodouracil, 5-chlorouracil, 5-(2-bromovinyl)uracil or 2-pyrimidione moiety, W¹ represents an oxygen or carbon atom or a methylidene group, W² represents a carbon atom or A¹, A² and W² jointly represent a sulphur or oxygen atom, Z¹ represents a hydrogen or fluorine atom or hydroxyl group, Z² represents a hydrogen or fluorine atom or hydroxyl or methyl group, or Z¹ with Z² jointly represent a fluoromethylene group, or A¹, A², Z¹ and Z² jointly represent a double bond, X represents an oxygen, sulphur or selenium atom, Y represents an oxygen or sulphur atom, R¹ represents a simple alkyl or aryl group with 1-6 carbon atoms or a moiety of a primary amino acid amide.

2. The process for the manufacture of 5′-O-[(N-acyl)amidophosphate]- and 5′-O-[(N-acyl)amidothiophosphate]- and 5″-O-[(N-acyl)amidodithiophosphate]- and 5′-O-[(N-acyl)amidoselenophosphate]- derivatives of nucleosides of general formula 1 wherein A¹ represents a fluorine atom or azide or hydroxyl group, A² represents a hydrogen atom, B¹ represents an adenine, 2-chloroadenine, 2-fluoroadenine, 2-bromoadenine, 2-iodoadenine, hypoxanthine, guanine, cytosine, 5-fluorocytosine, 5-bromocytosine, 5-iodocytosine, 5-chlorocytosine, azacytosine, thymine, 5-fluorouracil, 5-bromouracil, 5-iodouracil, 5-chlorouracil, 5-(2-bromovinyl)uracil or 2-pyrimidione moiety, W¹ represents an oxygen or carbon atom or a methylidene group, W² represents a carbon atom or A¹, A² and W² jointly represent a sulphur or oxygen atom, Z¹ represents a hydrogen or fluorine atom or hydroxyl group, Z² represents a hydrogen or fluorine atom or hydroxyl or methyl group, or Z¹ with Z² jointly represent a fluoromethylene group, or A¹, A², Z¹ and Z² jointly represent a double bond, X represents an oxygen, sulphur or selenium atom, Y represents an oxygen or sulphur atom, R¹ represents a simple alkyl or aryl group with 1-6 carbon atoms or a moiety of a primary amino acid amide characterized in that a nucleoside of general formula 2 wherein A², W¹ are as above, A³ represents a fluorine atom or azide or protected hydroxyl group, W² represents a carbon atom or A², A³ and W² jointly represent a sulphur atom, B² represents an adenine, 2-chloroadenine, 2-fluoroadenine 2-bromoadenine, 2-iodoadenine, hypoxanthine, guanine or cytosine moiety of formulas 3, 4, 5 wherein Z⁵ represents a hydrogen atom or a known exoamine blocking group, Z⁶ represents a hydrogen atom or a chlorine, fluorine, bromine or iodine atom, Z⁷ represents a hydrogen atom or fluorine, chlorine, bromine or iodine atom or B² represents a thymine moiety, an azacytosine moiety or a 5-fluorouracil, 5-chlorouracil, 5-bromouracil, 5-iodouracil, 5-(2-bromovinyl)uracil or 2-pyrimidione moiety, and Z³ represents a hydrogen or fluorine atom or a protected hydroxyl group, Z⁴ represents a hydrogen or fluorine atom, a protected hydroxyl group or a methyl group or Z³ and Z⁴ jointly represent a fluoromethylene group or A², A³, Z³, Z⁴ jointly represent a double bond, is condensed with a compound of general formula 6, wherein R², R³, R⁴ and R⁵ each represent a hydrogen atom, simple alkyl or aryl with 1-6 carbon atoms, R⁶ represents an aromatic or non-aromatic 5- or 6-membered heterocyclic ring with 1-3 heteroatoms selected from a group consisting of an oxygen atom, nitrogen atom and sulphur atom, wherein the aryl or heterocyclic groups may be substituted with 1, 2 or 3 substituents independently selected from a group consisting of alkyl, halogen atom, CHF₂, CF₃, alkoxyl, halogenoalkoxyl, alkylthio group or R⁶ represents alkyl or phenyl or cycloalkyl which may be substituted with 1, 2 or 3 substituents independently selected from a group consisting of alkyl, alkenyl, alkynyl, alkoxyl, alkenyloxyl, alkynoxyl, cycloalkyl, cycloalkenyl, cycloalkyloxyl, cycloalkenyloxyl, phenyl and halogen atom, and the phenyl may be substituted with 1-5 halogen atoms and/or 1-3 substituents independently selected from a group consisting of an alkyl, halogenoalkyl, alkoxyl, halogenoalkoxyl, alkylthio group or halogenoalkylthio group, wherein the phenylamide group may be condensed with a saturated or unsaturated 5- or 6-membered ring which may be substituted with one or more alkyl groups and/or possibly containing a heteroatom selected from a group consisting of an oxygen atom, a nitrogen atom and a sulphur atom, or R⁶ represents a moiety of a primary amino acid amide of general formula 7 wherein R⁷ represents an amino acid side chain, R⁸ represents a hydrogen atom or a known amino acid alpha-amine-blocking group, X represents an oxygen, sulphur or selenium atom, Y represents an oxygen or sulphur atom; the condensation is carried out in anhydrous organic solvents in the presence of condensation activators, and after reaction completion the amino acid alpha-amine-blocking group, the 2′- and 3′-hydroxyl blocking groups and the nucleoside exoamine blocking groups are removed using methods known in the art.

3. Method according to claim 2 characterized in that the protecting groups for the 2′- and 3′-hydroxyl groups include known protecting groups selected from a group consisting of the acyl, benzoyl, 4,4-dimethoxytriphenyl, benzyl, trialkylsilyl and in particular trimethylsilyl group.

4. Method according to claim 2 characterized in that the protecting groups used for the exoamine groups include known protecting groups selected from a group consisting of the phenoxyacetyl, isopropoxyacetyl, isobutyryl, benzoyl, (dialkylamino)methylene and (dialkylamino)ethylidene group.

5. Method according to claim 2 characterized in that the protecting groups used for the amino acid alpha-amine groups include known alpha-amine protecting groups selected from a group consisting of the acyl, trifluoroacetyl, 4,4-dimethoxytriphenyl, benzyloxycarbonyl and tert-butyloxycarbonyl group.

6. Method according to claim 2 characterized in that the condensation activators used include non-nucleophilic alcoholates, such as potassium tert-butanolate or amines, such as imidazole, 1-methylimidazole, 4-dimethylaminopyridine, triethylamine and in particular 1,8-diazabicyclo[5.4]undec-7-ene (DBU).

7. Method according to claim 2 characterized in that the condensation reaction is carried out in an anhydrous organic solvent selected from a group consisting of acetonitrile, methylene chloride, N,N-dimethylformamide, pyridine, dioxane and tetrahydrofurane.

8. Method according to claim 2 characterized in that a compound of formula 1, wherein X and Y represent an oxygen atom, is obtained from previously prepared compounds of formula 1 wherein X═S, Y═S or Y═O, or X═Se and Y═O in the oxidation reaction using an oxidation reagent known in the art, particularly hydrogen peroxide.

9. The process for the manufacture of 5′-O-[(N-acyl)amidophosphate]- and 5′-O-[(N-acyl)amidothiophosphate]- and 5′-O-[(N-acyl)amidodithiophosphate]- and 5′-O-[(N-acyl)amidoselenophosphate]-derivatives of nucleosides of general formula 1 wherein A¹ represents a fluorine atom or azide or hydroxyl group, A² represents a hydrogen atom, B¹ represents an adenine, 2-chloroadenine, 2-fluoroadenine, 2-bromoadenine, 2-iodoadenine, hypoxanthine, guanine, cytosine, 5-fluorocytosine, 5-bromocytosine, 5-iodocytosine, 5-chlorocytosine, azacytosine, thymine, 5-fluorouracil, 5-bromouracil, 5-iodouracil, 5-chlorouracil, 5-(2-bromovinyl)uracil or 2-pyrimidione moiety, W¹ represents an oxygen or carbon atom or a methylidene group, W² represents a carbon atom or A¹, A² and W² jointly represent a sulphur or oxygen atom, Z¹ represents a hydrogen or fluorine atom or hydroxyl group, Z² represents a hydrogen or fluorine atom or hydroxyl or methyl group, or Z¹ with Z² jointly represent a fluoromethylene group, or A¹, A2, Z¹ and Z² jointly represent a double bond, X represents an oxygen, sulphur or selenium atom, Y represents an oxygen or sulphur atom, R¹ represents a simple alkyl or aryl group with 1-6 carbon atoms or a moiety of a primary amino acid amide characterized in that the reagents used in the condensation include primary carboxylic amides of general formula R⁶CONH₂ wherein R⁶ is as above or amino acid amides with moieties of general formula 7 wherein R⁷ and R⁸ are as above with nucleoside derivatives of general formula 8 wherein A², A³, B², R², R³, R⁴, R⁵, W¹, W², Z³, Z⁴ are as above, X represents an oxygen, sulphur or selenium atom and Y represents an oxygen or sulphur atom; the condensation is carried out in anhydrous organic solvents in the presence of condensation activators, and after reaction completion the amino acid alpha-amine-blocking groups, the 2′- and 3′-hydroxyl blocking groups and the nucleoside exoamine blocking groups are removed using methods known in the art.

10. Method according to claim 9 characterized in that the protecting groups for 2′- and 3′-hydroxyl groups include known protecting groups selected from a group consisting of the acyl, benzoyl, 4,4-dimethoxytriphenyl, benzyl, trialkylsilyl and in particular trimethylsilyl group.

11. Method according to claim 9 characterized in that the protecting groups used for exoamine groups include known protecting groups selected from a group consisting of the phenoxyacetyl, isopropoxyacetyl, isobutyryl, benzoyl, (dialkylamino)methylene and (dialkylamino)ethylidene group.

12. Method according to claim 9 characterized in that the protecting groups used for amino acid alpha-amine groups include known alpha-amine protecting groups selected from a group consisting of the acyl, trifluoroacetyl, 4,4-dimethoxytriphenyl, benzyloxycarbonyl and tert-butyloxycarbonyl group.

13. Method according to claim 9 characterized in that the condensation activators used include non-nucleophilic alcoholates, such as potassium tert-butanolate or amines, such as imidazole, 1-methylimidazole, 4-dimethylaminopyridine, triethylamine and in particular 1,8-diazabicyclo[5.4]undec-7-ene (DBU).

14. Method according to claim 9 characterized in that the condensation reaction is carried out in an anhydrous organic solvent selected from a group consisting of acetonitrile, methylene chloride, N,N-dimethylformamide, pyridine, dioxane and tetrahydrofurane.

15. Method according to claim 9 characterized in that a compound of formula 1, wherein X and Y represent an oxygen atom, is obtained from previously prepared compounds of formula 1 wherein X═S, Y═S or Y═O, or X═Se and Y═O in an oxidation reaction using an oxidation reagent known in the art, particularly hydrogen peroxide.
